(19) United States
(12) Patent Application Publication
(10) Pub. No.: US 2003/0124721 A1
(43) Pub. Date: Jul. 3, 2003

(54) Endocrine pancreas differentiation of adipose tissue-derived stromal cells and uses thereof

(76) Inventors: Bentley Cheatham, Durham, NC (US); Jeffrey M. Gimble, Chapel Hill, NC (US); Yuan-Di C. Halvorsen, Branford, CT (US)

Correspondence Address: KING & SPALDING, 191 PEACHTREE STREET, N.E., ATLANTA, GA, 30303-1763, US

(21) Appl. No.: 10/293,398

(22) Filed: Nov. 12, 2002

Related U.S. Application Data
(60) Provisional application No. 60/344,913, filed on Nov. 9, 2001.

(51) Int. Cl. C12N 5/08; A61K 45/00

(52) U.S. Cl. 435/366; 424/93.7

(57) ABSTRACT

The invention provides cells, compositions and methods based on the differentiation of adipose tissue-derived stromal cells into a cell expressing at least one genotypic or phenotypic characteristic of a pancreas cell. The cells produced in the method are useful in providing a source of differentiated and functional cells for research, implantation, transplantation and development of tissue engineered products for the treatment of diseases of the pancreas and pancreatic tissue repair.

### CROSS REFERENCE TO RELATED APPLICATION

This application claims priority to U.S. Ser. No. 60/344,913 filed on Nov. 9, 2001.

### FIELD OF INVENTION

The invention provides isolated adipose tissue-derived stromal cells induced to express at least one characteristic of a pancreas cell. Methods for treating endocrine diseases of the pancreas are also provided.

### BACKGROUND OF INVENTION

The endocrine cell mass of the pancreatic islets of Langerhans is composed of four cell types, classified based on a major regulated secretory product. These include glucagon-producing α-cells, insulin-producing β-cells, pancreatic polypeptide-producing γ-cells and somatostatin-producing δ-cells (Henquin, 2000, Diabetes 49, 1751-1760; Slack, 1995, Development 121, 1569-1580). During development, these distinct cell populations are thought to arise from a common stem cell precursor associated with the pancreatic ductal epithelium (Rao et al., 1989, Am. J. Pathol. 134, 1069-1086; Rosenberg and Vinik, 1992, Adv. Exp. Med. Biol. 321:95-104; Swenne, 1992, Diabetologia 35, 193-201; Hellerstrom, 1984, Diabetologia 26, 393-400; Gu and Sarvetnick, 1993, Development 118, 33-46). The precursor cells, through a series of stepwise differentiation pathways, acquire properties of the various cell populations. Early observations indicate that the α-cells are the first detectable population of the islet followed sequentially by the γ-cells, δ-cells and γ-cells (Slack, 1995, Development 121, 1569-1580). The islet forms along the ductal epithelium as a mass of β-cells surrounded by α- or γ-cells and interdigitating δ-cells. The immature islet then migrates to surrounding acinar tissue and is vascularized (Slack, 1995, Development 121, 1569-1580).

A primary function of islet cells is physiologic nutrient homeostasis. For example, normally functioning β-cells synthesize and secrete insulin to maintain blood glucose levels. This is accomplished via an endogenous glucose-sensing apparatus that is linked to a secretory pathway for insulin's regulated release. In this stimulus-secretion coupling paradigm, elevated plasma glucose (e.g. post-prandial) alters β-cell metabolism resulting in alterations in membrane potential by closure of ATP-sensitive K+ channels. This depolarizing event opens voltage-sensitive Ca2+ channels and the influx of Ca2+ triggers the regulated release of insulin (Henquin, 2000, Diabetes 49, 1751-1760). The plasma insulin then acts to stimulate glucose uptake into skeletal muscle and adipose tissues, and inhibit hepatic glucose production with an overall result in lowering of plasma glucose (Cheatham and Kahn, 1995, Endocr. Rev. 16, 117-142).

I. Insulin

Type 1 (insulin-dependent) diabetes is a major disease associated with loss of endocrine pancreas function. In most cases this occurs by an autoimmune attack on the islets. Current therapy for Type 1 diabetes requires single to multiple daily injections of insulin. In the majority of cases this regime is not sufficient to maintain adequate control of blood glucose levels, resulting in numerous diabetic late complications, which greatly increase the rates of morbidity and mortality of the affected individuals. An alternative therapy to the daily insulin injections has been to cure diabetes through pancreatic or islet transplants (Serup et al., 2001, BMJ 322, 29-32; Soria et al., 2001, Diabetologia 44, 407-415). Studies indicate that although transplantation of intact pancreatic tissue is an effective treatment, this procedure suffers from three major obstacles: 1) shortage of donor material; 2) requirement of major surgical procedures and 3) the need for long-term immunosuppressive therapy with short-term benefits. Similarly, islet transplantation is only somewhat effective. This process involves isolation of islets from a donor pancreas and injection into the portal vein. Moreover, this procedure involves multiple injections requiring several hospitalizations (Serup et al., 2001, BMJ 322, 29-32; Soria et al., 2001, Diabetologia 44, 407-415). In addition, these patients must also undergo intensive immunosuppressive therapy. Furthermore, as in pancreatic tissue transplantation, isolated islet procedures also suffer from greatly limited donor populations. Studies using xenografted porcine islets are ongoing, however immune-rejection with this approach is still a significant barrier (Serup et al., 2001, BMJ 322, 29-32; Soria et al., 2001, Diabetologia 44, 407-415).

Taken together, the above data suggest the need for alternative cellular therapeutic approaches. Along these lines, both murine and human-derived pancreatic ductal stem cells and embryonic stem cells have been used to produce islet-like cell lines through controlled induction of differentiation down an endocrine pancreatic path (Assady et al., 2001, Diabetes 50, 1691-1697; Serup et al., 2001, BMJ 322, 29-32; Lumelsky et al., 2001, Science 292, 1389-1394; Soria et al., 2001, Diabetologia 44, 407-415). Murine-derived stem cells induced to differentiate into islet-hormone producing cells have been used successfully to reconstitute diabetic mouse-models (Serup et al., 2001, BMJ 322, 29-32; Soria et al., 2001, Diabetologia 44, 407-415). Again, barriers to these approaches include immune-rejection and greatly limited sources of precursor cell lines for application in humans.

Human embryonic stem cells (HES) have been successfully differentiated into cells which produce insulin (Assady et al., 2001, Diabetes 50, 1691-1697; Diabetes 50:1691-1697). The use of pleuripotent undifferentiated HES cells potentially represents a source of differential pancreatic beta cells which are utilized in diseases such as diabetes. However, these methods suffer from a number of disadvantages. First is the problem of a source of the HES cells themselves. Despite the recent publicity surrounding the critical need for research and development in the field of HES cells, political and ethical controversies remain. As a consequence, the availability of appropriate HES cells is not guaranteed. The second drawback of the use of HES cells in the production of differentiated pancreatic islet cells is the unpredictability of demonstrating glucose responsiveness in the cultured differentiated cells. Indeed, Assady and colleagues (Diabetes 50, 1691-1697) have suggested that cells differentiated from HES cells do not demonstrate glucose responsiveness. Unresponsiveness could be attributed to differences in the heterogeneity of the cell populations growing in the culture, the difficulty in normalizing insulin response to parameters such as protein or DNA content or long-term exposure to high glucose levels in culture. Thus in order to utilize differentiated β-cells, it is necessary to demonstrate that the differentiated cells possess stimulus-secretion coupling for insulin. HES cell therapies also suffer from the potential high risk of teratoma development.

The pancreas itself is the source of islet progenitor cells. WO01/23528 to the University of Florida Research Foundation disclose the use of islet progenitor cells grown in vitro for the implantation into a mammal for the in vivo therapy of diabetes.

Differentiation of pancreatic ductal-derived stem cells or isolated embryonic stem cells down the endocrine pancreatic lineages are characterized by expression of specific marker enzymes and transcription factors. Interestingly, during embryonic development islets and neural cells share many common markers including the neural-specific enolase, synaptophysins, catechol-synthesizing enzymes, tyrosine hydroxylase, nestin, and the transcription factors HNF3β, Isl-1, Brain-4 Pax-6, Pax-4, Beta2/NeuroD, Pancreatic and duodenal homeobox gene 1 (PDX-1), Nkx6.2, Nkx2.2 and neurogenin-3 (Ngn-3) (Ramiya et al., 2000, Nat. Med. 6, 278-282; Schwitzgebel et al., 2000, Development 127, 3533-3542; Fernandes et al., 1997, Endocrinology 138, 1750-1762; Zulewski et al., 2001, Diabetes 50, 521-533; Gradwohl et al., 2000, Proc. Natl. Acad. Sci. U.S.A 97, 1607-1611). Functional markers for more mature islet cells are the expression of glucagon, somatostatin, insulin, the glucose transporter 2 (Glut2) and pancreatic polypeptide.

II. Glucagon

Glucagon is a 29 amino acid peptide hormone liberated in the alpha cells of the islets of Langerhans. Glucagon-producing alpha cells represent one of the earliest populations of detectable islet cells in the developing endocrine pancreas. The tissue-specific liberation of proglucagon is controlled by cell-specific expression of prohormone convertase (PC) enzymes. An essential role for PC2 in the processing of islet proglucagon is revealed by studies of the PC2 knockout mouse. This mouse has mild hypoglycemia, elevated proinsulin, and exhibits a major defect in the processing of proglucagon to mature pancreatic glucagon, and the murine islet α cells secrete proglucagon from atypical secretory granules. (J Biol Chem. Feb. 6, 1998;273(6):3431-7; J Biol Chem. Jul. 20, 2001; 276(29):27197-202).

The key biological actions of glucagon converge on regulation of glucose homeostasis through enhanced synthesis and mobilization of glucose in the liver. Glucagon receptors are also expressed on human islet β cells and contribute to the regulation of glucose-stimulated insulin secretion (Diabetologia August 2000;43(8):1012-9).

Glucagon generally functions as a counter-regulatory hormone, opposing the actions of insulin, and maintaining the levels of blood glucose, particularly in patients with hypoglycemia. In patients with diabetes, excess glucagon secretion plays a primary role in the metabolic perturbations associated with diabetes, such as hyperglycemia. A major problem in diabetic patients with repeated hypoglycemia is the development of defective counter-regulatory responses that include reduced or absent glucagon responses to hypoglycemia. Hence understanding how and why the autonomic nervous system and islet α cells develop defects in glucagon secretion leading to hypoglycemia insensitivity is a major challenge in diabetes research.

Administration of glucagon pharmacologically leads to a rapid rise in blood glucose, hence injectable glucagon is used as a pharmacological treatment for diabetic patients at risk for significant hypoglycemia. Diabetes has long been viewed as a bihormonal disorder, with glucagon excess contributing significantly to the development of hyperglycemia. Shah and colleagues (Am. J. Physiol 1999 277:E283-E290) examined the importance of the ambient insulin concentration for development of glucagon-mediated hyperglycemia in human subjects following a prandial glucose load. The authors found that glucagon excess in the presence of relative insulin deficiency clearly contributes to impaired suppression of glucose production and hyperglycemia. Hence inhibitors of glucagon secretion or glucagon action may be useful for the treatment of diabetics with insulin deficiency and/or glucagon excess. Studies in patients with type 2 diabetes suggests that lack of glucagon suppression contributes to postprandial hyperglycemia in part via accelerated glycogenolysis. Analysis of blood glucose in the presence or absence of somatostatin-induced glucagon suppression during an oral glucose tolerance test (OGTT) revealed a significant increase in glucose in subjects with higher glucagon levels. (See J Clin Endocrinol Metab. November 2000;85(11):4053-9).

A number of studies have demonstrated that glucagon promotes degradation of fat (known as lipolysis) both in cell preparations and in vivo. Thus, glucagon may promote lipolysis in human adipose tissue. However, older studies had been contradictory, with some reports affirming a role for glucagon in human adipocyte lipolysis. Human glucagon and vasoactive intestinal polypeptide (VIP) stimulate free fatty acid release from human adipose tissue in vitro, whereas other experiments failed to show significant effects of glucagon on lipolysis in isolated human fat cells (Int. J. Obes. 1985;9(1):25-7). Glucagon withdrawal or physiological hyperglucagonemia in vivo did not produce significant changes in palmitate flux, an index of lipolysis, in normal or diabetic human subjects (J Clin Endocrinol Metab. February 1991;72(2):308-15). Similar negative findings were reported recently, wherein 7 healthy male subjects were implanted with indwelling microdialysis catheters in the abdominal wall, and the effects of glucagon infusion on interstitial glycerol, and plasma glycerol and FFAs were examined. No effects on glycerol or free fatty acids were detected with systemic glucagon infusion, with or without exogenous glucose. (J Clin Endocrinol Metab. May 1, 2001;86(5):2085-2089). Similar negative results were obtained in a study of lipolysis in normal male subjects with indwelling microdialysis catheters implanted into abdominal adipose tissue (J Clin Endocrinol Metab. May 2001;86(5):2085-9). Hence, the available data do not support an important physiological role for glucagon on lipolysis.

Glucagon has anti-motility effects on the gastrointestinal tract (esophagus, stomach, and small and large intestine) when administered pharmacologically to human subjects. (Dig Dis Sci. July 1979;24(7):501-8; Gut. December 1975;16(12):973-8; N Engl J Med. Nov. 11, 1999;341(20):1496-503). Glucagon may also relax smooth muscle in the gallbladder and ureter, leading to occasional use during radiology studies of the gallbladder and kidney.

III. Somatostatin

Somatostatin is an endogenous peptide produced in pancreatic delta cells that performs a variety of important functions within the body. Somatostatin is a highly flexible cyclic peptide with a very short biological half-life. Somatostatin, originally discovered to act as a classical endocrine hormone of the hypothalamic-pituitary system, has since been shown to act additionally as a paracrine and autocrine signaling factor on a wide variety of cell types. The numerous physiological processes currently recognized to be influenced by somatostatin include hormone and peptide factor secretion, neurotransmission, cell proliferation, smooth muscle contraction, nutrient absorption and inflammation. Hormones and peptides regulated by somatostatin include growth hormone (GH), thyroid-stimulating hormone (TSH), prolactin (PRL), insulin, and substance P (SP).

Somatostatin affects the function of many important biological systems such as the endocrine, gastrointestinal, vascular, and immune systems along with the central and peripheral nervous systems. In the endocrine system, somatostatin plays an important role in controlling growth hormone, insulin and glucagon secretion (Koerker et al., Science 1974, 184, 482-484). The effects of somatostatin on the gastrointestinal and vascular biological systems have led to clinical applications for somatostatin therapeutics in both of these areas. In the central nervous system (CNS), somatostatin appears to be an important regulator of cognitive functions (Schettini, Pharmacological Research 1991, 23, 203-215) and, in specific areas of the brain, appears to act as a neurotransmitter or as a neuromodulator regulating the release of neurotransmitters such as acetylcholine (Gray et al., J. of Neuroscience 1990, 10, 2687-2698) and dopamine (Thal et al., Brain Research 1986, 372, 205-209). In the peripheral nervous system (PNS), somatostatin is present in catecholamine containing fibers and in sensory terminals together with substance P (Green et al., Neuroscience 1992, 50, 745-749) and acts to inhibit their release and mediated effects.

Like somatostatin itself, somatostatin receptors have been localized to a wide variety of tissues and cell types including those belonging to the endocrine, gastrointestinal, vascular, immune, CNS, and PNS systems. A high incidence of somatostatin receptors has also been demonstrated in a variety of human tumors. Neuroendocrine tumors are one class of tumors that exhibit a high density of functionally active somatostatin receptors. Functionally active neuroendocrine tumors present with clinical symptoms such as gastrinoma and glucagoma syndrome due to excessive hormone release from the tumor cell. Such symptoms may be treated through somatostatin receptor activation.

IV. Pancreatic Polypeptide

Pancreatic gamma cells are known to secrete pancreatic polypeptide (PP) which is a member of the neuropeptide Y family of proteins. Little is known as to the precise physiological mechanism of this peptide. PP is known to exert effects directly in the pancreas by inhibiting the secretion of pancreatic digestive enzymes via inhibition on vagal nerve stimulation. This effect of PP is thought to occur through both a direct effect on the vagus as well as a central nervous system-mediated effect in the dorsal vagal complex and the arcuate nucleus (Deng et al Brain Res 2001; 902:18-29). Through its vagal nerve actions, PP is also thought to inhibit insulin release. PP also appears to inhibit the islet cell hypertrophy that is observed in non-insulin dependent diabetic conditions. Circulating levels of PP also exert an effect on the liver and lead to a decrease in hepatic glucose production. Thus, administration of PP may have a role in the treatment of non-insulin dependent diabetes mellitus.

V. Non-Embryonic Sources of Stem Cells

Adult cells have shown the ability for differentiation. For example, recent studies have demonstrated the specific ability of bone marrow-derived stromal cells to undergo neuronal differentiation in vitro (Woodbury et al. (2000) J Neuroscience Research 61:364; Sanchez-Ramos et al. (2000) Exp Neurology 164:247). In these investigations, treatment of bone marrow stromal cells with antioxidants, epidermal growth factor (EGF), or brain derived neurotrophic factor (BDNF) induced the cells to undergo morphologic changes consistent with neuronal differentiation, i.e., the extension of long cell processes terminating in growth cones and filopodia (Woodbury et al. (2000) J Neuroscience Research 61:364; Sanchez-Ramos et al. (2000) Exp Neurology 164:247). In addition, these agents induced the expression of neuronal specific protein including nestin, neuron-specific enolase (NSE), neurofilament M (NF-M), NeuN, and the nerve growth factor receptor trkA (Woodbury et al. (2000) J Neuroscience Research 61:364; Sanchez-Ramos et al. (2000) Exp Neurology 164:247

Other examples of adult cells having the ability for differentiation are described in the following patents:

U.S. Pat. No. 5,486,359 to Osiris is directed to an isolated, homogeneous population of human mesenchymal stem cells that can differentiate into cells of more than one connective tissue type. The patent discloses a process for isolating, purifying, and greatly replicating these cells in culture, i.e. in vitro.

U.S. Pat. No. 5,942,225 to Case Western and Osiris describes a composition for inducing lineage-directed differentiation of isolated human mesenchymal stem cells into a single particular mesenchymal lineage, which includes human mesenchymal stem cells and one or more bioactive factors for inducing differentiation of the mesenchymal stem cells into a single particular lineage.

U.S. Pat. No. 5,736,396 to Case Western describes a method of inducing ex vivo lineage-directed differentiation of isolated human mesenchymal stem cells which includes contacting the mesenchymal stem cells with a bioactive factor so as to thereby induce ex vivo differentiation thereof into a single particular mesenchymal lineage. The patent also describes a method of treating an individual in need of mesenchymal cells of a particular mesenchymal lineage which includes administering to an individual in need thereof a composition comprising isolated, human mesenchymal stem cells which have been induced to differentiate ex vivo by contact with a bioactive factor so as to thereby induce ex vivo differentiation of such cells into a single particular mesenchymal lineage.

U.S. Pat. No. 5,908,784 to Case Western discloses a composition for the in vitro chondrogenesis of human mesenchymal precursor cells and the in vitro formation of human chondrocytes therefrom, which composition includes isolated human mesenchymal stem cells condensed into close proximity as a packed cell pellet and at least one chondroinductive agent in contact therewith. The patent also describes a process for inducing chondrogenesis in mesenchymal stem cells by contacting mesenchymal stem cells with a chondroinductive agent in vitro wherein the stem cells are condensed into close proximity as a packed cell pellet.

U.S. Pat. No. 5,902,741 to Advanced Tissue Sciences, Inc. discloses a living cartilage tissue prepared in vitro, that includes cartilage-producing stromal cells and connective tissue proteins naturally secreted by the stromal cells attached to and substantially enveloping a three-dimensional framework composed of a biocompatible, non-living material formed into a three-dimensional structure having interstitial spaces bridged by the stromal cells. The patent also discloses a composition for growing new cartilage comprising mesenchymal stem cells in a polymeric carrier suitable for proliferation and differentiation of the cells into cartilage.

U.S. Pat. No. 5,863,531 to Advanced Tissue Sciences, Inc. discloses a tubular living stromal tissue prepared in vitro, comprising stromal cells and connective tissue proteins naturally secreted by the stromal cells attached to and substantially enveloping a three-dimensional tubular framework composed of a biocompatible, non-living material having interstitial spaces bridged by the stromal cells.

U.S. Pat. No. 6,022,743 to Advanced Tissue Sciences, Inc. describes a stromal cell based three-dimensional culture system derived form pancreatic parenchymal cells cultures on a living stromal tissue framework. The stromal cells can include umbilical cord cells, placental cells, mesenchymal stem cells or fetal cells. The culture system is thus used to provide functioning pancreatic tissue and organ material.

U.S. Pat. No. 5,811,094 to Osiris describes a method of producing a connective tissue that includes producing connective tissue in an individual in need thereof by administering to said individual a cell preparation containing human mesenchymal stem cells which is recovered from human bone marrow and which is substantially free of blood cells.

U.S. Pat. No. 6,030,836 to Thiede et al describes a method of maintaining human hematopoietic stem cells in vitro comprising co-culturing human mesenchymal stem cells with the hematopoietic stem cells such that at least some of the hematopoietic stem cells maintain their stem cell phenotype.

U.S. Pat. No. 6,103,522 to Torok-Storb et al describes an irradiated immortalized human stromal cell line in a combined in vitro culture with human hematopoietic precursor cells.

WO 9602662A1 and U.S. Pat. No. 5,879,940 to Torok-Storb et al describes human bone marrow stromal cell lines that sustain hematopoiesis.

U.S. Pat. No. 5,827,735 to Morphogen describes purified pleuripotent mesenchymal stem cells, which are substantially free of multinucleated myogenic lineage-committed cells, and which are predominantly stellate-shaped, wherein the mesenchymal stem cells form predominantly fibroblastic cells when contacted with muscle morphogenic protein in tissue culture medium containing 10% fetal calf serum and form predominantly branched multinucleated structures that spontaneously contract when contacted with muscle morphogenic protein and scar inhibitory factor in tissue culture with medium containing 10% fetal calf serum.

WO 99/43286 to Hahnemann University describes the use of mesenchymal stem cells to treat the central nervous system and a method of directing differentiation of bone marrow stromal cells.

WO 98/20731 to Osiris describes a mesenchymal megakaryocyte precursor composition and method of isolating MSCs associated with isolated megakaryocytes by isolating megakaryocytes.

WO 99/61587 to Osiris describes human CD45 and/or fibroblast and mesenchymal stem cells.

WO 01/079457 to Ixion Technology describes the use of bone marrow and blood-derived stem cells cultured and differentiated in vitro into pancreatic-like cells. WO 01/78752 to the University of Texas describes the use of neural stems implanted into a pancreas for the treatment of pancreatic disorders.

However, the techniques described in the preceding paragraphs rely on sources of precursor cells such as bone marrow that are difficult to obtain as well as being painful for the donor. Therefore, an object of the invention is to provide a cell, material and method to assist in the treatment of endocrine diseases of the pancreas.

### SUMMARY OF THE INVENTION

The present invention provides an isolated adipose tissue-derived stromal cell isolated from a human or other mammal induced to express at least one genotypic or phenotypic characteristic of a pancreas cell, and preferably, an endocrine pancreatic cell. The cell can exhibit a property of a glucagon-producing α-cell, insulin-producing β-cell, pancreatic polypeptide-producing γ-cell or a somatostatin-producing δ-cell. In a preferred embodiment, an insulin-producing β-cell is produced. The cell of the invention can be induced to differentiate in vitro or after implantation into a patient.

The cell of the invention can be incorporated into a two or three dimensional structure to create an implantable or implanted matrix, as described in more detail below. The present invention, for example, provides a method for encapsulating the differentiated adipose-derived adult stem cells or differentiated cells in a biomaterial compatible with transplantation into a mammal, preferably a human. The encapsulation material would not hinder the release of proteins or hormones secreted by the adipose-derived adult stem cells or differentiated cells. The materials used, include but are not limited to, collagen derivatives, hydrogels, calcium alginate, agarose, hyaluronic acid, poly-lactic acid/poly-glycolic acid derivatives and fibrin.

The cell of the invention can also be genetically engineered to include exogenous genetic material. In one embodiment, a vector is employed which is capable of integrating the desired gene sequences into the host cell chromosome. In a preferred embodiment, the introduced nucleic acid molecule is incorporated into a plasmid or viral vector capable of autonomous replication in the recipient host cell. Any of a wide variety of vectors can be employed for this purpose. Preferred eukaryotic vectors include for example, vaccinia virus, SV40, retroviruses, adenoviruses, adeno-associated viruses and a variety of commercially-available, plasmid-based mammalian expression vectors that are familiar to those experienced in the art. Once the vector or nucleic acid molecule has been prepared for expression, the DNA construct(s) can be introduced into an appropriate host cell by any of a variety of suitable means, i.e., transformation, transfection, viral infection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene molecule(s) results in the production of the heterologous protein.

The invention also provides for a method for differentiating isolated adipose tissue derived stromal cells to express at least one genotypic or phenotypic characteristic of a pancreas cell, for example, a glucagon producing α-cell, insulin-producing β-cell, pancreatic polypeptide-producing γ-cell or a somatostatin-producing δ-cell, comprising the step of: contacting an isolated adipose tissue-derived stromal cell with a pancreas inducing substance, preferably an endocrine pancreas inducing substance. This substance is in a chemically defined cell culture medium, as described in more detail below which can include growth factors, cytokines, chemical agents, and/or hormones at concentrations sufficient to induce isolated adipose tissue-derived stromal cells to express at least one endocrine pancreas cell marker.

The invention further provides a method of treating a disorder that is mediated by a pancreatic function of a glucagon-producing α-cell, insulin-producing β-cell, pancreatic polypeptide-producing γ-cell or a somatostatin-producing δ-cell, in a host that includes inducing an isolated adipose tissue-derived stromal cell to express at least one genotypic or phenotypic characteristic of the pancreas cell which is therapeutically beneficial to the host; and then transplanting the induced cells into the host. An advantage of the invention is that the adipose tissue-derived stromal cells can be isolated directly from the host, differentiated and then re-implanted autologously. Alternatively, the therapy can be accomplished allogeneically.

Non-limiting examples of pancreatic endocrine disorder or degenerative conditions that the current invention can be used to treat includes Type I Diabetes Mellitus, Type II Diabetes Mellitus, lipodystrophy associated disease, chemically-induced disease, pancreatitis-associated disease, or a trauma-associated disease.

The cell of the invention can be used either as a homogenous or substantially homogeneous population of cells or as part of a cell population in which the other cells secrete substances to support the growth or differentiation of the endocrine pancreas like cell or with other cells which secrete or exhibit other desired therapeutic factors.

The invention also includes methods of producing hormones via the treated adipose-derived stromal cells. Methods are also included for conditioning culture medium by exposing a cell culture medium to the cell of the invention. The medium can then be used to culture other adipose-derived cells.

The invention also contemplates a kit for producing adipose derived-stromal cells that have been induced to express at least one genotypic or phenotypic characteristic of a pancreas cell, that can include instructions for separating the stromal or stem cells from the remainder of the adipose tissue, and does include a medium for differentiating the stem cells, wherein the medium causes the cell to express at least one genotypic or phenotypic characteristic of a pancreas cell, or is generally pancreogenic. A kit is also disclosed that includes all the necessary components to create the tissue of the invention. Such a kit includes the cell or cell population of the invention, the biologically compatible lattice, as well as components consisting of hydrating agents, cell culture substrates, cell culture media, other cells, antibiotic compounds, and hormones.

Other objects and features of the invention will be more fully apparent from the following disclosure.

### DETAILED DESCRIPTION OF THE INVENTION

The present invention provides an isolated adipose tissue-derived stromal cell from a human or other mammal induced to express at least one genotypic or phenotypic characteristic of a pancreas cell, and preferably, an endocrine pancreatic cell. The cell can exhibit a property of a glucagon-producing α-cell, insulin-producing β-cell, pancreatic polypeptide-producing γ-cell or a somatostatin-producing δ-cell. In a preferred embodiment, an insulin-producing β-cell is produced. The cell of the invention can be induced to differentiate in vitro or after implantation into a patient.

The cells produced by the methods of invention can provide a source of partially or fully differentiated, functional cells having characteristics of mature pancreatic cells for research, transplantation, and development of cellular therapeutic products for the treatment of animal diseases, preferably human diseases, tissue repair or improvement, and the correction of life-altering or life-threatening metabolic disorders. Methods to produce such cells are also included.

I. Definitions

“Developmental phenotype” is the potential of a cell to acquire a particular physical phenotype through the process of differentiation.

“Genotype” is the expression of at least one messenger RNA transcript of a gene associated with a differentiation pathway.

“Pancreatic beta islet cell” is intended to mean any cell capable of secreting insulin, insulin analogue, insulin precursor or an insulin-like factor, preferably in a regulated manner, more preferably in a glucose concentration-dependent manner.

“Pancreatic alpha cell” is intended to mean any cell capable of secreting glucagon, glucagon analogue, glucagon precursor or a glucagon-like factor, preferably in a regulated manner.

“Pancreatic delta cell” is intended to mean any cell capable of secreting somatostatin, somatostatin precursor or a somatostatin-like factor, preferably in a regulated manner.

By “pancreatic PP cell” or “gamma cell” is intended any cell capable of secreting pancreatic peptide, analogue, precursor or a similar factor, preferably in a regulated manner.

By “insulin” is intended any of the various insulin, insulin analogues or insulin-like factors known. This includes the prohormone or insulin precursor proteins, the fully processed protein, or a metabolite of any of these entities.

“Diabetes mellitus” is intended to mean any disease state where pancreatic beta islet cell function is dysfunctional such that there is a loss of responsiveness to circulating glucose levels. The disease state may be a consequence of inborn metabolic error, traumatic injury, chemical injury, infectious disease, chronic alcohol ingestion, endocrinopathies, genetic disorders such as Down's Syndrome, or any other etiology causing damage directly or indirectly to the endocrine pancreas.

“Mature onset diabetes of the young” is meant to include the small percentage of diabetic patients that do not fall clearly into either the type 1 or type 2 diabetes phenotype. It is characterized by a genetic defect in beta-cell function with an early onset, usually before age 25.

“Autoimmune disease” is intended to encompass any immune mediated process, humoral or cellular, that results in the rejection and destruction of the host's endocrine pancreas. The etiology of this process is, but is not limited to, immune response to an infection by an agent such as coxsackie virus, or Mycoplasma pneumoniae, inborn metabolic propensity to autoimmune dysfunction, or a chemical exposure.

Polyacrylamide Gel Electrophoresis (PAGE). The most commonly used technique (though not the only one) for achieving a fractionation of polypeptides on the basis of size is polyacrylamide gel electrophoresis. The principle of this method is that polypeptide molecules migrate through the gel as though it were a sieve that retards the movement of the largest molecules to the greatest extent and the movement of the smallest molecules to the least extent. The smaller the polypeptide fragment, the greater the mobility under electrophoresis in the polyacrylamide gel. Both before and during electrophoresis, the polypeptides typically are continuously exposed to the detergent sodium dodecyl sulfate (SDS), under which conditions the polypeptides are denatured. Native gels are run in the absence of SDS. The polypeptides fractionated by polyacrylamide gel electrophoresis can be visualized directly by a staining procedure.

Western Transfer Procedure. The purpose of the western transfer procedure (also referred to as immunoblotting) is to physically transfer polypeptides fractionated by polyacrylamide gel electrophoresis onto a nitrocellulose filter or another appropriate surface, while retaining the relative positions of polypeptides resulting from the fractionation procedure. The blot is then probed with an antibody that specifically binds to the polypeptide(s) of interest.

A “purified” protein or hormone is a protein or hormone that has been separated from a cellular component. “Purified” proteins or hormones have been purified to a level of purity not found in nature. A “substantially pure” protein or hormone is a protein or hormone is a preparation that contains only other components that do not materially affect the properties of the hormone or protein.

“Genes of the endocrine pancreas” is intended to include but not be limited to any of those genes associated with the phenotype of the differentiating or differentiated alpha, beta, delta or PP cells of the endocrine pancreas. These genes include but, are not limited to, pdx1, pax4, pax6, neurogenin 1, neurogenin 2, neurogenin 3, neuro D, GLUT2, insulin, Isl1, Hlxb9, Nkx2.2.

II. Adipose-Derived Stem or Stromal Cells

Adipose stem cell or “adipose stromal cell” refers to cells that originate from adipose tissue. By “adipose” is meant any fat tissue. The adipose tissue may be brown or white adipose tissue, derived from subcutaneous, omental/visceral, mammary, gonadal, or other adipose tissue site. Preferably, the adipose is subcutaneous white adipose tissue. Such cells may comprise a primary cell culture or an immortalized cell line. The adipose tissue may be from any organism having fat tissue. Preferably, the adipose tissue is mammalian, most preferably the adipose tissue is human. A convenient source of adipose tissue is from liposuction surgery, however, the source of adipose tissue or the method of isolation of adipose tissue is not critical to the invention. Liposuction is a relatively non-invasive procedure with cosmetic effects, which are acceptable to the vast majority of patients. It is well documented that adipocytes are a replenishable cell population. Even after surgical removal by liposuction or other procedures, it is common to see a recurrence of adipocytes in an individual over time at the same site. This suggests that adipose tissue contains stromal stem cells, which are capable of self-renewal into adipocytes.

Pathologic evidence suggests that adipose-derived stromal cells are capable of differentiation along multiple lineage pathways. The most common soft tissue tumors, liposarcomas, develop from adipocyte-like cells. Soft tissue tumors of mixed origin are relatively common. These may include elements of adipose tissue, muscle (smooth or skeletal), cartilage, and/or bone. In patients with a rare condition known as progressive osseous heteroplasia, subcutaneous adipocytes form bone for unknown reasons.

Human adipose tissue-derived adult stromal cells can be expanded ex vivo, differentiated along unique lineage pathways, genetically engineered, and re-introduced into individuals as either autologous or allogeneic transplantation.

WO 00/53795 to the University of Pittsburgh and The Regents of the University of California and U.S. patent application Ser. No. 2002/0076400 assigned to the University of Pittsburgh, disclose adipose-derived stem cells and lattices substantially free of adipocytes and red blood cells and clonal populations of connective tissue stem cells. The cells can be employed, alone or within biologically-compatible compositions, to generate differentiated tissues and structures, both in vivo and in vitro. Additionally, the cells can be expanded and cultured to produce hormones and to provide conditioned culture media for supporting the growth and expansion of other cell populations. In another aspect, these publications disclose a lipo-derived lattice substantially devoid of cells, which includes extracellular matrix material form adipose tissue. The lattice can be used as a substrate to facilitate the growth and differentiation of cells, whether in vivo or in vitro, into anlagen or mature tissue or structures. Neither publication discloses adipose tissue derived stromal cells that have been induced to express at least one phenotypic or genotypic characteristic of a endocrine pancreas cell.

U.S. Pat. No. 6,391,297 assigned to Artecel Sciences discloses a composition of an isolated human adipose tissue-derived stromal cell that has been differentiated to exhibit at least one characteristic of an osteoblast that can be used in vivo to repair bone and treat bone diseases. This adipose-derived osteoblast-like cell can be optionally genetically modified or combined with a matrix.

U.S. Pat. No. 6,426,222 assigned to BioHoldings International discloses methods for inducing osteoblast differentiation from human extramedullary adipose tissue by incubating the adipose tissue cells in a liquid nutrient medium that must contain a glucocorticoid.

WO 00/44882 and U.S. Pat. No. 6,153,432 listing Halvorsen et al as inventors, discloses methods and compositions for the differentiation of human preadipocytes isolated from adipose tissue into adipocytes bearing biochemical, genetic, and physiological characteristics similar to that observed in isolated primary adipocytes.

WO 01/62901 and published U.S. patent application Ser. No. 2001/0033834 to Artecel Sciences discloses isolated adipose tissue-derived stromal cells that have been induced to express at least one phenotypic characteristic of a neuronal, astroglial, hematopoietic progenitor or hepatic cell. In addition, an isolated adipose tissue-derived stromal cells that has been dedifferentiated such that there is an absence of adipocyte phenotypic markers is also disclosed.

U.S. Pat. No. 6,429,013 assigned to Artecel Sciences discloses compositions directed to an isolated adipose tissue-derived stromal cell that has been induced to express at lease one characteristic of a chondrocyte. Methods are also disclosed for differentiating these cells.

U.S. Pat. No. 6,200,606 to Peterson et al., discloses that precursor cells which have the potential to generate bone or cartilage can be isolated from a variety of hematopoetic and non-hematopoetic tissues including peripheral blood, bone marrow and adipose tissue.

The adipose tissue derived stromal cells useful in the methods of invention are isolated by a variety of methods known to those skilled in the art such as described in WO 00/53795 to the University of Pittsburgh et al. and WO 00/44882 and U.S. Pat. No. 6,153,432 to Zen-Bio, Inc. In a preferred method, adipose tissue is isolated from a mammalian subject, preferably a human subject. A preferred source of adipose tissue is subcutaneous adipose. In humans, the adipose is typically isolated by liposuction. If the cells of the invention are to be transplanted into a human subject, it is preferable that the adipose tissue be isolated from that same subject so as to provide for an autologous transplant. Alternatively, the transplanted tissue may be allogenic.

As a non-limiting example, in one method of isolating adipose tissue derived stromal cells, the adipose tissue is treated with collagenase at concentrations between 0.01 to 0.5%, preferably 0.04 to 0.2%, most preferably 0.1%, trypsin at concentrations between 0.01 to 0.5%, preferably 0.04 to 0.04%, most preferably 0.2%, at temperatures between 25° to 50° C., preferably between 33° to 40° C., most preferably at 37° C., for periods of between 10 minutes to 3 hours, preferably between 30 minutes to 1 hour, most preferably 45 minutes. The cells are passed through a nylon or cheesecloth mesh filter of between 20 microns to 800 microns, more preferably between 40 to 400 microns, most preferably 70 microns. The cells are then subjected to differential centrifugation directly in media or over a Ficoll or Percoll or other particulate gradient. Cells are centrifuged at speeds of between 100 to 3000×g, more preferably 200 to 1500×g, most preferably at 500×g for periods of between 1 minutes to 1 hour, more preferably 2 to 15 minutes, most preferably 5 minutes, at temperatures of between 4° to 50° C., preferably between 20° to 40° C., most preferably at 25° C.

In yet another method of isolating adipose-derived stromal cells a mechanical system such as described in U.S. Pat. No. 5,786,207 to Katz et al is used. A system is employed for introducing an adipose tissue sample into an automated device, subjecting it to a washing phase and a dissociating phase wherein the tissue is agitated and rotated such that the resulting cell suspension is collected into a centrifuge-ready receptacle. In such a way, the adipose-derived cells are isolated from a tissue sample, preserving the cellular integrity of the desired cells.

III. Inducement of Adipose-Derived Stromal Cells to Exhibit at Least One Characteristic of a Pancreas Cell

The invention includes the treatment of the adipose-derived stromal cells to induce the formation of a cell that expresses at least one genotypic or phenotypic characteristic of a pancreatic cell. Non-limiting examples of how to induce the differentiation of adiposederived stromal cells include: 1) the use of cell media; 2) the use of support cells; 3) direct implantation of the undifferentiated cells into the tissue of a patient; and 4) cellular engineering techniques.

A) Cell Media Inducement

While the invention is not bound by any theory of operation, it is believed that treatment of the adipose-derived stromal cells with a medium containing a combination of serum, embryonic extracts, purified or recombinant growth factors, cytokines, hormones, and/or chemical agents, in a 2-dimensional or 3-dimensional microenvironment, will induce differentiation.

More specifically, the invention provides a method for differentiating an adipose-derived cells into a cell having a genotypic or phenotypic property of a pancreatic cell, comprising: plating isolated adipose-derived adult stem cells at a desired density, including but not limited to a density of about 1,000 to about 500,000 cells/cm²; incubating the cells in a chemically defined culture medium comprising at least one compound selected from the group consisting of: growth factor, hormone, cytokine, serum factor, nuclear hormone receptor liquid, or any other defined chemical agent.

Base media useful in the methods of the invention include, but are not limited to, Neurobasal™ (supplemented with or without, fetal bovine serum or basic fibroblastic growth factor (bFGF)), N2, B27, Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's sale base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA-with non essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. A preferred medium for use in the present invention is DMEM. These and other useful media are available from GIBCO, Grand Island, N.Y., USA and Biological Industries, Beth Aemek, Israel, among others. A number of these media are summarized in Methods in Enzymology, Volume LVIII, “Cell Culture”, pp. 62-72 (ed. Jakoby and Pastan, Academic Press, Inc).

Media useful for the differentiation of adipose-derived stromal cells into cells that express at least one genotypic or phenotypic characteristic of a pancreatic beta cell includes secretin or any secretin analogue or agonist that have been shown to be important in the differentiation of progenitor cells into insulin-secreting beta cells as disclosed in WO 00/47721 to Ontogeny, Inc. et al. Media useful in the methods of the invention will contain fetal serum of bovine or other species origin at a concentration of at least 1% to about 30%, preferably at least about 5% to 15%, mostly preferably about 10%. Embryonic extract of chicken or other species origin is present at a concentration of about 1% to 30%, preferably at least about 5% to 15%, most preferably about 10%.

The growth factors, cytokines, hormones used in the invention including, but are not limited to, growth hormone, erythropoeitin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor, fibroblast growth factor, nerve growth factor, cilary neurotrophic factor, platelet derived growth factor, and bone morphogenetic protein at concentrations of between picogram/ml to milligram/ml levels. For example at such concentrations, the growth factors, cytokines and hormones useful in the methods of the invention are able to induce, up to 100% the formation of blood cells (lymphoid, erythroid, mycloid or platelet lineages) from adipose derived stromal cells in colony forming unit (CFU) assays. (Moore et al. (1973) J. Natl. Cancer Inst. 50:603-623; Lee et al. (1989) J. Immunol. 142:3875-3883; Medina et al. (2993) J. Exp. Med. 178:1507-1515.

Growth factors which have been shown to be able to assist in producing insulin-producing cells include the peptides, GLP-1, extendin-4 as well as analogues having substantially homologous amino acid sequences as disclosed in WO 00/09666 to Egan et al.

It is further recognized that additional components may be added to the culture medium. Such components may be antibiotics, albumin, amino acids, and other components known to the art for the culture of cells. Additionally, components may be added to enhance the differentiation process. Other chemical agents can include, but are not limited to, steroids, retinoids, and other chemical compounds or agents that induce the differentiation of adipose derived stromal cells by at least 25-50% relative to a positive control.

It is recognized that the cell media conditions described above yields a cell that expresses at least one genotypic or phenotypic characteristic of a single type of pancreas cell, i.e. pancreatic alpha, beta, delta or PP cell. The particular cell types are separated by any means known to those skilled in the art. Particularly useful are means that take advantage of the genotypic or phenotypic characteristics expressed by the differentiated cells. Phenotypic markers of the desired cells as listed below are well known to those of ordinary skill in the art, and copiously published in the literature. Additional phenotypic markers continue to be disclosed or can be identified without undue experimentation. Any of these markers are used to confirm that the adipose-derived adult stem cells have been induced to a differentiated state.

Lineage specific phenotypic characteristics can include, but are not limited to, cell surface proteins, cytoskeletal proteins, cell morphology, and/or secretory products. Pancreatic alpha cells express glucagons, among other markers. Pancreatic beta islet cell characteristics include the expression of markers including but not limited to nestin, pdx1 (also known as IDX-1, IPF-1 and STF-1), GLUT2, NeuroD, neurogenin, and insulin. Furthermore, pancreatic beta-cells contain large amounts of zinc. Use of a non-toxic zinc-sensitive fluorescent probe will selectively label labile zinc in viable beta-cells and exhibit excitation and emission wavelengths in the visible spectrum, making this technique exploitable by standard instrumentation (Lukowiak B et al., J Histochem Cytochem. April 2001;49(4):519-28). Cell sorting of dissociated Newport Green-labeled cells resulted in a clear separation of beta-cells, as judged by insulin content per DNA and immunocytochemical analysis. By use of flow cytometry or similar cell sorting tools, one skilled in the art will be able to purify beta cells in high purity using this or a comparable technique.

Pancreatic delta cells express somatostatin, among other markers while pancreatic PP cells express pancreatic polypeptide. Other markers for these cell-types include: receptor for cholecystokinin (CCKA) and KIT receptor tyrosine kinase (Schweiger et al Anat Histol Embryol. December 2000;29(6):357-61; Rachdi et al Diabetes September 2001;50(9):2021-8).

An alternative method uses antibodies directed specifically to markers found on the various cell types for purification via many well-documented techniques known to those skilled in the art. These techniques include, but are not limited to, immunochemical flow cytometry and cell sorting and immunomagnetic purification. A non-limiting example of immunomagnetic purification involves the use of dynabeads which are uniform, paramagnetic particles coated with specific antibodies (i.e. insulin, glucagon, somatostatin or pancreatic polypetide).

One of ordinary skill in the art will recognize that known calorimetric, fluorescent, immunochemical, polymerase chain reaction, chemical or radiochemical methods can readily ascertain the presence or absence of a lineage specific marker.

In another embodiment, the invention provides a dedifferentiated, isolated, adipose-derived adult stem cell capable of being induced to express at least one genotypic or phenotypic characteristic of a pancreatic cell within a culture medium capable of such differentiation. A dedifferentiated adipose-derived adult stem cell is identified by the absence of mature adipocyte markers.

B) Use of Support Cells to Promote the Differentiation of the Adipose-Derived Stromal Cells

In another embodiment of the invention, support cells are used to promote the differentiation of the adipose-derived stromal cells. The support cells can be human or nonhuman-animal derived cells. If nonhuman-animal support cells are used, the resulting differentiated cells are implanted via xenotransplantation.

Adipose-derived cells of the invention are isolated and cultured within a population of cells, most preferably the population is a defined population. The population of cells is heterogeneous and includes support cells for supplying factors to the cells of the invention. Support cells include other cell types that will promote the differentiation, growth and maintenance of the desired cells. As a non-limiting example, if an adipose-derived stromal cell that expresses at least one genotypic or phenotypic characteristic of a pancreatic beta cell is desired, adipose-derived stromal cells are first isolated by any of the means described above, and grown in culture in the presence of other support cells. For example, these support cells preferably possess the characteristic of other pancreatic cell types, i.e. alpha, delta, gamma, PP. In another embodiment, the support cells are derived from primary cultures of these cell types taken from cultured pancreas tissue. In yet another embodiment, the support cells are derived from immortalized cell lines. In some embodiments, the support cells are obtained autologously. In other embodiments, the support cells are obtained allogeneically.

It is also contemplated by the present invention that the cells used to support the differentiation of the desired cell can be genetically engineered to be support cells. The cells are genetically modified to express exogenous genes or to repress the expression of endogenous genes by any method described below or know to those skilled in the art.

C) Implantation

In another aspect, the invention provides adipose-derived stromal cells and differentiated cells expressing at least one genotypic or phenotypic characteristic of a pancreas cell that is useful in autologous and allogenic transplantations. The differentiation takes place in vivo by means of factors naturally in the environment or introduced factors. In one embodiment, the site of transplantation is a diseased pancreas. In other embodiments the site of transplantation is subcutaneous or intraperitoneal. Preferably, the subject is mammalian, more preferably, the subject is human. In another embodiment, the cell is implanted in an area that is in need of glucagon, pancreatic polypeptide, somatostatin, or insulin with or without additional growth factors. The cell of the invention can be induced to differentiate in vitro or after implantation into a patient.

Thus, in still another aspect, the invention discloses a method for providing differentiated cells of expressing at least one a genotypic or phenotypic characteristic of a pancreas cell to a subject, comprising:

a) isolating adipose tissue-derived stromal cells;

b) plating and incubating the cells in a medium appropriate for the differentiation of the cells;

c) introducing the differentiated cells into the subject.

In another embodiment of the invention, a method for providing undifferentiated adipose-derived stromal cells to a subject, comprising:

a) isolating adipose tissue-derived stromal cells;

b) introducing the undifferentiated cells into the subject.

It is contemplated for in the invention that when undifferentiated adipose-derived stromal cells are introduced into the subject, in one particular embodiment, they are introduced directly into a diseased pancreas with or without additional growth or differentiation factors. In yet another aspect of the invention, the undifferentiated adipose-derived stromal cells are introduced along with any of the support cells or differentiation factors as described herein that will provide an environment suitable for the in vivo differentiation of the stromal cells. For example, these support cells preferably possess the characteristic of other pancreatic cell types, i.e. alpha, beta, delta, gamma, PP. In another embodiment, the support cells are derived from primary cultures of these cell types taken from cultured pancreas tissue. In yet another embodiment, the support cells are derived from immortalized cell lines. In some embodiments, the support cells are obtained autologously. In other embodiments, the support cells are obtained allogeneically.

In another embodiment, the dedifferentiated adipose-derived cell is provided in combination with a pharmaceutically acceptable carrier for a therapeutic application, including but not limited to tissue repair, regeneration, reconstruction or enhancement. Adipose-derived cells are cultured by methods disclosed in U.S. Pat. No. 6,153,432 (herein incorporated by reference) to dedifferentiate the cells such that the dedifferentiated adult stem cells can then be induced to express genotypic or phenotypic characteristics of cells other than adipose tissue derived cells. The dedifferentiated adipose-derived cells are modified to include a non-endogenous gene sequence for production of a desired protein or peptide. The dedifferentiated adipose-derived cell can, in an alternative embodiment, be administered to a host in a two- or three-dimensional matrix for a desired therapeutic purpose. In one embodiment, the dedifferentiated cell is obtained autologously from the patient's own cells. Alternatively, the dedifferentiated cell is obtained allogeneically.

In still another aspect of the invention, the differentiated cells of the invention are disaggregated and transferred to suspended cell culture suspensions similar to the methods disclosed in WO 97/15310 to the University of Florida Research Foundation, which discloses methods for the in vitro growth of functional islets of Langerhans from pancreatic tissue-derived stem cells, and grown until evidence of islet cell cluster formation is observed. Media containing the clusters can then be analyzed by standard biochemical analytical techniques known to those skilled in the art for the presence of insulin or other hormones that would be indicative of pancreatic endocrine function. The clusters or aggregates can then be injected or engrafted into the host tissue for tissue generation or regeneration purposes.

Encapsulation

The present invention provides a method for encapsulating the differentiated adipose-derived cells in a biomaterial compatible with transplantation into a mammal, preferably a human. The encapsulation material should be selected not hinder the release of desired proteins secreted by the adipose-derived adult stem cells. The materials used include but are not limited to collagen derivatives, hydrogels, calcium alginate, agarose, hyaluronic acid, poly-lactic acid/poly-glycolic acid derivatives and fibrin.

D) Genetic Manipulation of the Adipose-Derived Cells of the Invention

In yet another embodiment, the adipose-tissue derived cell expressing at least one genotypic or phenotypic characteristic of a pancreas cell is genetically modified to express exogenous genes or to repress the expression of endogenous genes. The invention provides a method of genetically modifying such cells and populations.

A nucleic acid construct comprising a promoter and the sequence of interest can be introduced into a recipient prokaryotic or eukaryotic cell either as a non-replicating DNA (or RNA) molecule, which can either be a linear molecule or, more preferably, a closed covalent circular molecule. Since such molecules are incapable of autonomous replication without an origin of replication, the expression of the gene can occur through the transient expression of the introduced sequence. Alternatively, permanent expression can occur through the integration of the introduced DNA sequence into the host chromosome.

In one embodiment, a vector is employed which is capable of integrating the desired gene sequences into the host cell chromosome. Cells that have stably integrated the introduced DNA into their chromosomes can be selected by also introducing one or more markers which allow for selection of host cells which contain the desired nucleic acid sequence. The marker, if desired, can provide for prototrophy to an auxotrophic host, biocide resistance, e.g., resistance to antibiotics, or heavy metals, such as copper, or the like. The selectable marker gene sequence can either be directly linked to the DNA gene sequences to be expressed, or introduced into the same cell by co-transfection. Preferably, expression of the marker can be quantified and plotted linearly.

In a preferred embodiment, the introduced nucleic acid molecule is incorporated into a plasmid or viral vector capable of autonomous replication in the recipient host. Any of a wide variety of vectors can be employed for this purpose. Factors of importance in selecting a particular plasmid or viral vector include: the ease with which recipient cells that contain the vector can be recognized and selected from those recipient cells which do not contain the vector; the number of copies of the vector which are desired in a particular host; and whether it is desirable to be able to “shuttle” the vector between host cells of different species.

Preferred eukaryotic vectors include for example, vaccinia virus, SV40, retroviruses, adenoviruses, adeno-associated viruses and a variety of commercially-available, plasmid-based mammalian expression vectors that are familiar to those experienced in the art.

Once the vector or nucleic acid molecule containing the construct(s) has been prepared for expression, the DNA construct(s) can be introduced into an appropriate host cell by any of a variety of suitable means, i.e., transformation, transfection, viral infection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene molecule(s) results in the production of the heterologous protein.

Introduced DNA being “maintained” in cells should be understood as the introduced DNA continuing to be present in essentially all of the cells in question as they continue to grow and proliferate. That is, the introduced DNA is not diluted out of the majority of the cells over multiple rounds of cell division. Rather, it replicates during cell proliferation and at least one copy of the introduced DNA remains in almost every daughter cell. Introduced DNA may be maintained in cells in either of two fashions. First, it may integrate directly into the cell's genome. This occurs at a rather low frequency. Second, it may exist as an extrachromosomal element, or episome. In order for an episome not to be diluted out during cell proliferation, a selectable marker gene can be included in the introduced DNA and the cells grown under conditions where expression of the marker gene is required. Even in the case where the introduced DNA has integrated in the genome, a selectable marker gene may be included to prevent excision of the DNA from the chromosome.

The genetically altered cells can be introduced into an organism by a variety of methods under conditions for the transgene to be expressed in vivo. Thus, in a preferred embodiment of the invention, the transgene can encode for the production of insulin. The cells containing the transgene for insulin can then be introduced into the pancreas of a diseased human or other mammal. Alternatively, the cells containing the transgene are injected intraperitoneally or into some other suitable organ depot site.

E) Cellular Characterization

By “characterization” of the resulting differentiated cells is intended the identification of surface and intracellular proteins, genes, and/or other markers indicative of the lineage commitment of the stromal cells to a particular terminal differentiated state. These methods can include, but are not limited to, (a) detection of cell surface proteins by immunofluorescent methods using protein specific monoclonal antibodies linked using a secondary fluorescent tag, including the use of flow cytometric methods; (b) detection of intracellular proteins by immunofluorescent methods using protein specific monoclonal antibodies linked using a secondary fluorescent tag, including the use of flow cytometric methods; (c) detection of cell genes by polymerase chain reaction, in situ hybridization, and/or northern blot analysis.

Terminally differentiated cells may be characterized by the identification of surface and intracellular proteins, genes, and/or other markers indicative of the lineage commitment of the stromal cells to a particular terminal differentiated state. These methods, which are described above, include, but are not limited to, (a) detection of cell surface proteins by immunofluorescent assays such as flow cytometry or in situ immunostaining of adipose-derived stromal cells surface proteins such as alkaline phosphatase, CD44, CD146, integrin beta 1 or osteopontin (Gronthos et al. 1994 Blood 84:4164-4173) insulin, glucagon, somatostatin, pancreatic polypeptide, nestin, PDX1, GLUT2, neuroD, and neurogenin; (b) detection of intracellular proteins by immunofluorescent methods such as flow cytometry or in situ immunostaining of adipose tissue-derived stromal cells using specific monoclonal antibodies; (c) detection of the expression of lineage selective mRNAs such as HNF3β, Isl-1, Brain-4, Pax-6, Pax-4, Beta2/NeuroD, PDX-1, Nkx6-2, Ngn-3, insulin and Glut-2 by methods such as polymerase chain reaction, in situ hybridization, and/or other blot analysis (See Gimble et al. 1989 Blood 74:303-311).

F) Use of the Cells of the Invention as Therapeutic Agents

The cells and populations of the present invention can be employed as therapeutic agents. Generally, such methods involve transferring the cells to the desired tissue or depot. The cells are transferred to the desired tissue by any method appropriate, which generally will vary according to the tissue type. For example, cells can be transferred to a graft by bathing the graft or infusing it with culture medium containing the cells. Alternatively, the cells can be seeded on the desired site within the tissue to establish a population. Cells can be transferred to sites in vivo using devices well know to those skilled in the art for example, catheters, trocars, cannulae, or stents seeded with the cells etc.

The cells of the invention find use in therapy for a variety of disorders. Particularly, disorders associated with endocrine dysfunction of the pancreas are of interest, or disorders that can be treated with glucagon, insulin, pancreatic polypeptide or somatostatin.

i) Insulin-Related Disorders

The transformed cells may be used to treat any insulin-related disorder such as diabetes mellitus, particularly Type I Diabetes Mellitus, Type II Diabetes Mellitus and Maturity-Onset Diabetes of the Young (MODY). The diabetic conditions that the cells of the invention are used to treat can arise from any etiology including but not limited to, genetic, infection, trauma, or chemical. Other diseases that are treated by the cells of the invention include lipodystrophy-associated disease, chemically-induced disease, pancreatitis-associated disease, or a trauma-associated disease. The disease state can be the result, for example, of an autoimmune dysfunction or infection by a virus or some other infectious agent.

ii) Glucagon-Related Disorders

Glucagon producing cells can be used for the treatment of: chronically hypoglycemic patients as implants that release glucagon systemically or on demand; irritable bowel syndrome or similar conditions that require smooth muscle relaxation; and obesity by stimulation of lipolysis by glucagon in fat cells to reduce adipose tissue mass.

iii) Pancreatic-Polypeptide-Related Disorders

Cells secreting pancreatic-polypeptide can be implanted and used for the treatment of: non-insulin dependent diabetes mellitus, obesity, or any condition that results in the hypertrophy of pancreatic beta islet cells, insulin resistance, or abnormal glucose production by the liver.

iv) Somatostatin-Related Disorders

The transformed cells can be used to treat any disorder for which somatostatin administration is helpful. Thus, the invention contemplates that the differentiated cells that express at least one characteristic of a pancreatic delta (i.e. somatostatin-producing) cell are used for the treatment and prevention of disorders wherein somatostatin itself, or the physiological processes it regulates, are involved. These include disorders of the endocrine, gastrointestinal, CNS, PNS, vascular and immune systems as well as cancer. Thus the differentiated somatostatin-producing cells are used: 1) to inhibit various hormone secretions and trophic factors in mammals; 2) to treat disorders involving, for example, autocrine or paracrine secretions of trophic factors including cancers of the breast, brain, prostate, and lung (both small cell and non-small cell epidermoids), as well as hepatomas, neuroblastomas, colon and pancreatic adenocarcinomas (ductal type), chondrosarcomas, and melanomas. In one embodiment, somatostatin-producing cells of the present invention are used to treat cancer directly or sensitize cancer cells for combination treatments using other regimens including radiation therapy or chemotherapy.

Other uses of these cells also include suppressing certain endocrine secretions, such as, insulin, glucagon, prolactin, and GH, which in turn can further suppress the secretion of various trophic factors such as IGF-1. The cells of the invention are accordingly indicated for use in the treatment of disorders with an etiology comprising or associated with excess GH and trophic factor secretion. The ability to suppress these secretions is useful in the treatment of disorders such as acromegaly. This activity is also useful in the treatment of neuroendocrine tumors, such as carcinoids, VIPomas, insulinomas and glucagonomas. The somatostatin-producing cells of this invention are also useful for treating diabetes and diabetes-related pathologies, including angiopathy, dawn phenomenon, neuropathy, nephropathy, and retinopathy (Grant et al., Diabetes Care 2000, 23, 504-509).

In another embodiment, somatostatin-producing cells of the subject invention are used to treat vascular disorders including bleeding disorders of the gastrointestinal system, such as those involving the splanchnic blood flow and esophageal varices associated with diseases such as cirrhosis. The ability of somatostatin of to mediate vasoconstriction also renders the somatostatin-producing cells useful in the treatment of cluster headache and migraine.

The somatostatin-producing cells of the invention can also be used to inhibit the proliferation of vascular endothelial cells and so are indicated for use in treating graft vessel diseases such as restenosis or vascular occlusion following vascular insult such as angioplasty, allo- or xenotransplant vasculopathies, graft vessel atherosclerosis, and in the transplantation of an organ (e.g., heart, liver, lung, kidney or pancreatic transplants (Weckbecker et al., Transplantation Proceedings 1997, 29, 2599-2600)). The somatostatin-producing cells of the invention can also be used to inhibit angiogenesis and are indicated for use in wound healing and treating metastatic stage cancer including but not limited to lung, breast and prostate cancers.

The somatostatin-producing cells of the subject invention can also be used for inhibiting gastric and exocrine and endocrine pancreatic secretion and the release of various peptides of the gastrointestinal tract. Thus, the somatostatin-producing cells are useful in treating gastro-intestinal disorders, for example in the treatment of peptic ulcers, NSAID-induced ulcers, ulcerative cholitis, acute pancreatitis (e.g., in post-ERCP patients), enterocutaneous and pancreaticocutaneous fistula, disturbances of GI motility, intestinal obstruction, chronic atrophic gastritis, non-ulcer dyspepsia, scleroderma, irritable bowel syndrome, Crohn's disease, dumping syndrome, watery diarrhea syndrome, and diarrhea associated such diseases as AIDS or cholera (see O'Dorisio et al., Advances in Endocrinology Metabolism 1990,1: 175-230).

In a specific embodiment, the somatostatin-producing cells disclosed herein are also functional where somatostatin is required as a neuromodulator in the central nervous system, with useful applications in the treatment of neurodegenerative diseases such as stroke, multiple sclerosis, Alzheimer's disease and other forms of dementia, mental health disorders (such as anxiety, depression, and schizophrenia), and in other neurological diseases such as pain and epilepsy (A. Vezzani et al., European Journal of Neuroscience 1999, 11, 3767-3776).

The somatostatin-producing cells can also be used in combination with other therapeutic agents. For example, in the case of treating organ transplantation, examples of other therapeutic agents include cyclosporin and FK-506. For treating tumors, examples of other agents include tamoxifen and alpha-interferon. For diabetes, examples of other compounds include metformin or other biguanides, acarbose, sulfonylureas thiazolidinediones or other insulin sensitizers including, but not limited to, compounds which function as agonists on peroxisome proliferator-activated receptor gamma (PPAR-gamma), insulin, insulin-like-growth factor I, glucagon-like peptide I (glp-I) and available satiety-promoting agents such as dexfenfluramine or leptin.

III. Tissue Entineering

The cells described herein can be employed in tissue engineering. The invention provides methods for producing animal matter comprising maintaining the inventive cells under conditions sufficient for them to expand and differentiate to the desired matter. The matter can include, for example a portion of, or even a whole pancreas. As such, the cells described herein are used in combination with any known technique of tissue engineering, including but not limited to those technologies described in the following: U.S. Pat. Nos. 5,902,741 and 5,863,531 to Advanced Tissue Sciences, Inc.; U.S. Pat. No. 6,139,574, Vacanti et al.; U.S. Pat. No. 5,759,830, Vacanti et al.; U.S. Pat. No. 5,741,685, Vacanti,; U.S. Pat. No. 5,736,372, Vacanti et al.; U.S. Pat. No. 5,804,178, Vacanti et al.; U.S. Pat. No. 5,770,417, Vacanti et al., U.S. Pat. No. 5,770,193, Vacanti et al.; U.S. Pat. No. 5,709,854, Griffith-Cima et al., U.S. Pat. No. 5,516,532, Atala et al.; U.S. Pat. No. 5,855,610, Vacanti et al.; U.S. Pat. No. 5,041,138, Vacanti et al.; U.S. Pat. No. 6,027,744, Vacanti et al.; U.S. Pat. No. 6,123,727, Vacanti et al., U.S. Pat. No. 5,536,656, Kemp et al.; U.S. Pat. No. 5,144,016, Skjak-Braek et al.; U.S. Pat. No. 5,944,754, Vacanti; U.S. Pat. No. 5,723,331, Tubo et al.; and U.S. Pat. No. 6,143,501, Sittinger et al..

To produce such a structure, the inventive cells and populations are maintained under conditions suitable for them to expand and divide to form the organ. This may be accomplished by transferring them to an animal typically at a sight at which the new matter is desired. Thus, the invention can facilitate the regeneration of a pancreas within an animal where the cells are implanted into such tissues.

In still other embodiments, the cells are induced to differentiate and expand into tissue in vitro. As such, the cells are cultured on substrates that facilitate formation into three-dimensional structures conducive for tissue development. Thus, for example, the cells are cultured or seeded on to a bio-compatible lattice, such as one that includes extracellular matrix material, synthetic polymers, cytokines, growth factors, etc. Such a lattice can be molded into desired shapes for facilitating the development of tissue types.

Thus, the invention provides a composition comprising the cells and populations and a biologically compatible lattice. The lattice can be formed from polymeric material, having fibers as a mesh or sponge, typically with spaces on the order of between 100 μm and about 300 μm. Such a structure provides sufficient area on which the cells can grow and proliferate. Desirably, the lattice is biodegradable over time, so that it will be absorbed into the animal matter as it develops. Suitable polymers can be formed from monomers such as glycolic acid, lactic acid, propyl fumarate, caprolactone, and the like. Other polymeric material can include a protein, polysaccharide, polyhydroxy acid, polyorthoester, polyanhydride, polyphosphozene, or a synthetic polymer, particularly a biodegradable polymer, or any combination thereof. Also, the lattice can include hormones, such as growth factors, cytokines, morphogens (e.g. retinoic acid etc), desired extracellular matrix materials (e.g. fibronectin, laminin, collagen etc) or other materials (e.g. DNA, viruses, other cell types etc) as desired.

To form the composition, the cells are introduced into the lattice such that they permeate into interstitial spaces therein. For example, the matrix can be soaked into a solution or suspension containing the cells, or they can be infused or injected in the matrix. Preferably, a hydrogel formed by cross-linking of a suspension including the polymer and also having the inventive cells dispersed therein is used. This method of formation permits the cells to be dispersed throughout the lattice, facilitating more even permeation of the lattice with the cells. Of course, the composition also can include mature cells of a desired phenotype or precursors thereof, particularly to potentiate the induction of the incentive cells within the lattice or promote the production of hormones such as insulin or glucagon within the lattice.

Those skilled in the art will appreciate that lattices suitable for inclusion into the composition can be derived from any suitable source, e.g. matrigel, and can of course include commercial sources for suitable lattices. Another suitable lattice can be derived from the acelluar portion of adipose tissue for example adipose tissue extracellular matrix substantially devoid of cells. Typically such adipose-derived lattices include proteins such as proteoglycans, glycoproteins, hyaluronin, fibronectins, collagens and the like, all of which serve a excellent substrates for cell growth. Additionally, such adipose-derived lattices can include hormones, cytokine, growth factors and the like. Those skilled in the art would be aware of methods for isolating such an adipose-derived lattice such as that disclosed in WO 00/53795 to the University of Pittsburgh, incorporated herein by reference.

In yet another embodiment of the invention, pancreatic-like tissue is created using solid free-form fabrication methods to allow for tissue regeneration and growth. Such techniques are disclosed, for example, in U.S. Pat. No. 6,138,573 to Vacanti et al and allow the creation of partial or whole organs for implantation into a human in need thereof. In particular, these techniques will allow for the creation of a partial or whole pancreas for implantation. Creation of such partial or whole organs is accomplished with the cells of the present invention obtained in an autologous manner. Alternatively, such partial or whole organs are created from cells of the invention that were obtained in an allogeneic manner. It is contemplated that any method known to those skilled in the art is useful for engineering tissue from the cells of the invention. For example, U.S. Pat. Nos. 6,022,743 and 5,516,681 to Naughton et al (Advanced Tissue Sciences) disclose methods for 3-dimensional cell culture systems for the culture of pancreatic-like tissue. These techniques involve the seeding and implanting of cells onto a matrix to form organ tissue and structural components that can additionally provide controlled release of bioactive agents. The matrix is characterized by a network of lumens functionally equivalent to the naturally occurring vasculature of the tissue formed by the implanted cells and that is further lined with endothelial cells. The matrix is further coupled to blood vessels or other ducts at the time of implantation to form a vascular or ductile network throughout the matrix. The free-form fabrication techniques refer to any technique known in the art that builds a complex 3-dimensional object as a series of 2-dimensional layers. The methods can be adapted for use with a variety of polymeric, inorganic and composite materials to create structures with defined compositions, strengths and densities. Thus, utilizing such methods, precise channels and pores can be created within the matrix to control subsequent cell growth and proliferation within the matrix of one or more cells types having a defined function. In such a way, differentiated cells of the present invention, corresponding to the various types of pancreas cells (i.e. cells possessing at least genotypic or phenotypic characteristic of a pancreas alpha, beta, gamma or delta cell) can be combined to form a partial or whole organ. Such cells are combined in the matrix to provide a vascular network lined with endothelial cells interspersed throughout the cells. Other structures can also be formed for use as lymph ducts, bile and other exocrine or excretory ducts within the organ.

The cells, populations, lattices and compositions of the invention are used in tissue engineering and regeneration. Thus, the invention pertains to an implantable structure incorporating any of the disclosed inventive features. The exact nature of the implant will vary according to the use desired. The implant can comprise mature tissue or can include immature tissue or the lattice. Thus for example, an implant can comprise a population of the inventive cells that are undergoing or pancreatic differentiation, optionally seeded within a lattice of a suitable size and dimension. Such an implant is injected or engrafted within a host to encourage the generation or regeneration of mature pancreatic tissue within the patient.

The adipose-derived lattice is conveniently employed as part of a cell culture kit. Accordingly, the invention provides a kit including the inventive adipose-derived lattice and one or more other components, such as hydrating agents (e.g. water, physiologically-compatible saline solutions, prepared cell culture media, serum or combinations or derivatives thereof), cell culture substrates (e.g. dishes, plates vials etc), cell culture media (whether in liquid or powdered form), antibiotics, hormones and the like. While the kit can include any such ingredients, preferably it includes all ingredients necessary to support the culture and growth of the desired cells upon proper combination. The desired kit can also include cells that are seeded into the lattice as described.

The present invention now will be described more fully by the following examples. This invention may, however, be embodied in many different forms and should not be construed as limited to the embodiments set forth herein; rather, these embodiments are provided so that this disclosure will be thorough and complete, and will fully convey the scope of the invention to those skilled in the art.

### EXAMPLES

### Example 1

### In Vitro Inductive Methods

Adipose-derived stem cells are isolated from liposuction waste material as described (Sen et al., 2001, J. Cell Biochem. 81, 312-319). These cells are continued in culture in the presence of (but not limited to) the following media: Neurobasal™ (In Vitrogen) supplemented with or without fetal bovine serum (FBS), N2, B27 (InVitrogen), or basic fibroblastic growth factor (bFGF). Modulation of glucose levels in the media is performed. Cells are seeded at various densities and fed at intervals of every 3-6 days. Most preferably, they are seeded at a density of about 1000 to about 500,000 cells/cm².

During the culture period, conditioned media is analyzed using commercially available radio-immunoassays or enzyme-linked immunosorbent assays for the endocrine pancreatic hormones insulin (American Laboratory Products), glucagon, somatostatin, and pancreatic polypeptide (Peninsula Labs Inc).

Expression of phenotypic markers associated with the differentiation of various endocrine pancreas cell lineages are assessed by analysis of mRNA by RT-PCR using specific primers for the following (but not limited to) genes: HNF3β, Isl-1, Brain-4, Pax-6, Pax-4, Beta2/NeuroD, PDX-1, Nkx6.2, Nkx2.2, Ngn-3, insulin, and Glut2. The presence of these markers and their association with endocrine pancreas cells has been previously described (Ramiya et al., 2000, Nat. Med. 6, 278-282; Schwitzgebel et al., 2000, Development 127, 3533-3542; Fernandes et al., 1997, Endocrinology 138, 1750-1762; Zulewski et al., 2001, Diabetes 50, 521-533; Gradwohl et al., 2000, Proc. Natl. Acad. Sci. U.S.A 97, 1607-1611). Immunohistochemical (IHC) analysis will also be performed using antibodies against (but not limited to) any of the above described phenotypic markers.

### Example 2

### Gene Therapy Methods

This method includes the insertion and expression of any gene that results in the induction of an adult stem cell to differentiate into a cell expressing at least one genotypic or phenotypic characteristic of a pancreas cell. These genes may include but are not limited to the controlled expression of the transcription factors HNF3β, Isl-1, Brain-4, Pax-6, Pax-4, Beta2/NeuroD, PDX-1, Nkx6.2, Nkx2.2 and Ngn-3. Potential methods for introducing nucleic acids into the cells include, but are not limited to, electroporation, calcium phosphate, retroviral, adenoviral or lipid-mediated delivery as described in detail above. Cells are analyzed for differentiation as described in detail above and in Example 1.

### Example 3

### In Vivo Transplantation

The cells of the present invention are implanted in vivo for therapeutic use in animals and in the treatment of human disorders resulting from malfunction of endocrine pancreas tissues, such as Type 1 diabetes. Existing rodent models for these applications include the insulin-dependent non-obese diabetic (NOD) mouse, and mice or rats rendered diabetic through destruction of islets by treatment with streptozotocin (Lumelsky et al., 2001, Science 292, 1389-1394; Soria et al., 2001, Diabetologia 44, 407-415). NOD mice have been used for implantation of pancreatic islets and islets produced from pancreatic ductal stem cells (Soria et al., 2001, Diabetologia 44, 407-415; Lumelsky et al., 2001, Science 292, 1389-1394; Stegall et al., 2001). Differentiated cells of the present invention that express at least one genotypic or phenotypic characteristic of a pancreas cell are used for implantation into the NOD animal, which normally must be maintained on daily insulin injections for survival. Preparation of the animals for implant includes a surgical procedure in which a small channel is created in the subcapsular region of a kidney capsule using a 27-gauge needle as previously described (Ramiya et al., 2000, Nat. Med. 6, 278-282). A starting range of 10³-10⁶ endocrine pancreas cells derived from human adult stem cells are implanted using a small catheter and the opening to the channel cauterized. An alternative procedure can be examined in which a subcutaneous site on the shoulder are prepared and the cells implanted (Ramiya et al., 2000, Nat. Med. 6, 278-282). In both of these implant models, sham operated NOD mice and NOD mice not undergoing any procedure serve as controls. Over a 2-7 day period following the surgical procedure the animals are weaned from the daily insulin injections. As an index for functional insulin-producing cells, animals are monitored for blood glucose levels at various stages using an AccuChek-EZ glucose monitor (Roche). ELISA assays for human insulin and other endocrine pancreas hormones are performed. In addition, immunohistochemical analysis of the implant sites are performed using human specific antibodies against insulin and other proteins potentially produced from the implant.

### Example 4

### Encapsulation

Cells implanted as described above can be immuno-rejected. In an effort to combat this, methods have been designed using encapsulating matrices that allow passage of secreted hormones from the encapsulated tissue, but serve as a protective barrier against a host immune attack. See Ramiya et al., 2000, Nat. Med. 6:278:282. Such barriers can include the hyaluronic acid-based gel Restalyne™ (Q-Med Sweden, Uppsala, Sweden). For this approach, a starting range of 10³-10⁶ endocrine pancreas cells derived from human adult stem cells are encapsulated into the gel. The implant placed in a subcutaneous site, animals weaned from insulin and analyzed as described above in Example 3.

### Example 5

### Allogeneic Transplantation

One example of an animal model for examining allogeneic transplantation has been described (Stegall et al., 2001, Transplantation 71, 1549-1555). Two strains (Strain A and Strain B; e.g. CBA (H-2k) and BALB/c (H2-d) of mice are used as adult stem cell donors and as recipients of endocrine pancreas cells derived from adult stem cells. The donor endocrine pancreas cells are produced from Strain A or Strain B adult stem cells isolated from a donor population of murine gonadal adipocytes in an analogous manner as described above for human adipose-derived stem cells. Recipients for Strain A or Strain B are rendered diabetic by treatment with streptozotocin (Stegall et al., 2001, Transplantation 71, 1549-1555). Transplants are established such that donorrecipient combinations are: 1) Isogeneic, Strain A to Strain A and Strain B to Strain B; 2) Allogeneic, Strain A to Strain B and Strain B to Strain A; 3) A third model using streptozoticin-induced diabetic nude (immunodeficient) mice as a recipient for donor cells from Strain A or Strain B. Animals receiving transplants are monitored and analyzed as described in Example 3.

### Example 6

### Insulin Detection Assay

In any of the cell cultures of the current invention, production of insulin is detected as follows. Briefly, cells grown as in any of the above examples are washed three times with serum-free medium containing 5 to 25 mmol/l glucose and incubated in 3 ml of serum-free medium for at least 2 hours. Subsequently, conditioned media is collected, and insulin levels are measured using a microparticle enzyme immunoassay (AXSYM™ system Insulin kit code B2D010; Abbott Laboratories) that detects human insulin with no cross-reactivity to proinsulin or C-peptide.

### Example 7

### Islet Cluster Formation

Three-dimensional islet clusters are constructed, for example, according to the method of Lumelsky et al (2001, Science 1389-1394). Briefly, cells are cultured according to the methods outlined in Example 1 described herein. Cells are initially grown to produce a highly enriched population of nestin-positive cells in suspension and are supplemented with serum-free ITSFn media. These conditions have been shown to increases the proportion of nestin-positive cells. Cells are then expanded in the presence of bFGF in N2 serum-free medium. To induce differentiation and morphogenesis of insulin secreting islet cluster, the bFGF is then withdrawn from the media that contains B27 media supplement with nicotinamide. The resulting aggregates or clusters are then be identified and verified as insulin-producing by any means known to those skilled in the art.

### Example 8

### Isolation of Specific Pancreatic Cell Types Differentiated from Adipose-Derived Stromal Cells

Single rat islet cells were initially incubated with the beta-cell surface specific antibody (K14D10 mouse IgG) for 20-60 min. A suspension of Dynabeads coated with a secondary antibody (anti-mouse IgG) was added for a further 15 min, after which the Dynabead-coated cells were instantaneously pelleted by contact between the tube and a magnet (Dynal MPC). Immunocytochemistry was used to confirm that the Dynabead-coated cells contained insulin and to quantify the efficiency of the method. Dynabead-coated and non-coated cells are stained for insulin and glucagon.

Dynabead immunopurification yielded 95% pure insulin-containing beta cells, which release insulin in response to isobutylmethylxanthine and glucagon-like polypeptide 1. The insulin content of Dynabead-coated beta cells is significantly higher than that of non-coated cells. Successful separation is achieved using as few as 30 islets as starting material. Using the “comet” assay, Dynabead-coated beta cells show equal susceptibility to cytokine-induced DNA damage as non-coated cells (Hadjivassiliou et al Diabetologia. September 2000;43(9):1170-7).

Modifications and other embodiments of the invention will be apparent to one skilled in the art to which this invention pertains having the benefit of the teachings presented in the foregoing descriptions. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

We claim:

1. An isolated adipose tissue-derived stromal cell induced to express at least one characteristic of endocrine pancreas-derived cell.

2. The cell of claim 1, wherein the cell is induced to differentiate in vitro.

3. The cell of claim 1, wherein the cell is induced to differentiate in vivo.

4. The cell of claim 1, wherein exogenous genetic material has been introduced into the cell.

5. The cell of claim 1, wherein the cell is human.

6. The cell of claim 1, wherein the cell secretes a hormone.

7. The cell of claim 6, wherein the hormone is selected from the group of hormones consisting of insulin, glucagon, somatostatin, or pancreatic polypeptide

8. The cell of claim 7, wherein the hormone is insulin

9. A method for differentiating isolated adipose tissue-derived stromal cells to display at least one endocrine pancreas cell marker comprising contacting an isolated adipose tissue-derived stromal cell with an endocrine pancreas inducing substance.

10. The method of claim 9, wherein the endocrine pancreas inducing substance is in a chemically defined cell culture medium.

11. A method of treating a pancreatic endocrine disorder or degenerative condition in a host comprising:
i) inducing isolated adipose tissue-derived stromal cells to express at least one endocrine pancreas cell marker; and
ii) transplanting the induced cells into the host.

12. The method of claim 11, wherein the adipose tissue-derived stromal cells are isolated from the host.

13. The method of claim 11, wherein the pancreatic endocrine disorder or degenerative condition is Type I Diabetes Mellitus, Type II Diabetes Mellitus, lipodystrophy associated disease, a chemically-induced disease, a pancreatitis-associated disease, or a trauma-associated disease.

14. An implant comprising the cells of any of claims 1-8.

15. The implant of claim 14 further comprising a biocompatible polymer.

16. The implant of claim 15, wherein the biocompatible polymer is a hydrogel.

17. The implant of claim 15, wherein the biocompatible polymer is collagen-derived, polyglycolic acid, polylactic acid, polyglycolic/polylactic acid, hyaluronate or fibrin.

18. A method of producing hormones, comprising (a) culturing the cell of any of claims 1-8 within a medium under conditions sufficient for the cell to secrete the hormone into the medium and (b) isolating the hormone from the medium.

19. The method of claim 18, wherein the hormone produced is selected from the group consisting of insulin, glucagon, somatostatin or pancreatic polypeptide.

20. The method of claim 19, wherein the hormone produced is insulin.

21. An isolated adipose tissue-derived stromal cell which is dedifferentiated in vitro and then induced to express at least one characteristic of an endocrine pancreas cell.

22. The dedifferentiated cell of claim 21 induced in vivo to express at least one characteristic of an endocrine pancreas cell.

23. An isolated adipose tissue-derived cell induced in culture to express at least one characteristic of an endocrine pancreas cell, wherein the induced cell is:
a) disaggregated and transferred to suspended cell cultures;
b) the suspended cell cultures are grown until evidence of islet cell cluster formation is observed; and, wherein
c) the resulting islet cell clusters are engrafted into a host.

24. The cell of claim 1 further encapsulated in a biomaterial compatible with transplantation into a host.

25. The cell of claim 24, wherein the encapsulation material is selected from the group consisting of collagen derivatives, hydrogels, calcium alginate, agarose, hyaluronic acid, poly-lactic acid/poly-glycolic acid derivatives and fibrin.

26. The use of the cell of any of claims 1-8, or 21-25 implanted into a host.

27. The use of the implant of any of claim 14-17 engrafted into a host.
